(19) United States
(12) Patent Application Publication
(10) Pub. No.: US 2024/0277582 A1
(43) Pub. Date: Aug. 22, 2024

(54) FREE-RADICALLY POLYMERIZABLE COMPOSITIONS FOR 3D PRINTING OF DENTAL CROWNS, BRIDGES, PROSTHETIC TEETH OR FULL PROSTHESES

(71) Applicant: VOCO GmbH, Cuxhaven (DE)

(72) Inventors: Sarah Metzscher, Hagen (DE); Daniel Oldenburger, Cuxhaven (DE); Manfred Thomas Plaumann, Cuxhaven (DE)

(21) Appl. No.: 18/442,764

(22) Filed: Feb. 15, 2024

(30) Foreign Application Priority Data
Feb. 16, 2023 (DE) 10 2023 103 800.1

(51) Int. Cl. A61K 6/887; A61C 5/77; A61C 13/00; A61C 13/087; A61K 6/836; A61K 6/853; B33Y 40/20; B33Y 70/10; B33Y 80/00

(52) U.S. Cl. CPC A61K 6/887; A61C 5/77; A61C 13/0019; A61C 13/087; A61K 6/836; A61K 6/853; B33Y 40/20; B33Y 70/10; B33Y 80/00

(57) ABSTRACT

The present invention relates to free-radically polymerizable compositions for 3D printing of dental crowns, bridges, prosthetic teeth or full prostheses which on account of their filler combination feature very good mechanical properties and optimal sedimentation stability.

### CROSS-REFERENCE TO RELATED APPLICATIONS

This application claims priority to German Patent Application No. 102023103800.1, filed Feb. 16, 2023, which is herein incorporated by reference in its entirety.

### BACKGROUND OF THE DISCLOSURE

The present invention relates to free-radically polymerizable compositions for 3D printing of dental crowns, bridges, prosthetic teeth or full prostheses.

The production of dental restorations, such as crowns, bridges, prosthetic teeth or full prostheses, today still predominantly employs conventional processes comprising a plurality of time-intensive steps. A subtractive CAD/CAM process is often also employed to produce dental restorations. This comprises milling the desired restoration from a prefabricated CAD/CAM block. This method too is costly and inconvenient and the milling of the dental restoration results in large material losses, with the result that costs are accordingly high.

In recent years additive manufacturing processes, for example stereolithography, have gained in importance in dentistry and thus also for the production of dental restorations.

In stereolithography suitable resin systems are subjected to layerwise curing with an ultraviolet laser. Generally, classical (meth)acrylate resins provided with suitable photoinitiators are polymerized. A vertically movable platform having a suitable surface (metal, glass, ceramic, etc.) for growing the solid resin phase is lowered to a distance of one layer thickness (20-200 um) measured from the bottom of the liquid resin bath. This is followed by a locally selective crosslinking and curing of the resin by a laser which is guided into the resin bath from below using micromirrors, for example. After the crosslinking of a first resin layer the solid polymer that forms on the platform is moved upwards in the resin bath by a further layer thickness and is thus reoccupied with liquid resin. The laser then cures the second layer. The sequence of raising the movable platform endowed with resin layers and the subsequent polymerizing are repeated until the entire three-dimensional workpiece has been constructed.

Stereolithographic processes inter alia have the advantage that they are less time-intensive and more precise than the conventional production processes of crowns, bridges, prosthetic teeth or full prostheses. It is also possible to produce a plurality of restorations in one pass. In contrast to the subtractive CAD/CAM process material losses in stereolithography are much lower since the uncured material may be reused.

For stereolithography, it is usually advantageous when the employed resin has a low viscosity. This is generally achieved by employing a low filler proportion. However, the consequence of a low filler proportion is that the required mechanical properties, for example strength and abrasion resistance, often do not meet the requirements for dental restorations.

It is thus necessary for the production of dental restorations using stereolithography to provide a composition which meets the respective requirements both in the unpolymerized state and after polymerization as the finished product.

Compositions for 3D printing of dental restorations are known from the prior art.

WO 2014/078537 A1 (Dentsply) discloses a composition for production of dental products by 3D printing, comprising (meth)acrylate, inorganic filler, organic filler, “silico-acrylic-based rubber impact modifier”, pigments and photoinitiators.

WO 2017/223084 A1 (Dentsply) claims a dental product (for example tooth prosthesis, prosthesis base) which is produced by 3D printing and consists of at least two layers comprising different compositions. The first layer comprises an oligomer, a polymerizable acrylic compound, “a rubber impact modifier” and a photoinitiator. The second layer contains a urethane monomer, a glycol dimethacrylate, a filler and a photoinitiator.

WO 2019/048963 A1 (3M) describes a printable composition having a viscosity of less than 150 Pas comprising at least one radiation-curable component, a photoinitiator and 50% by weight of a filler having an average particle size of 0.3 um. The composition is suitable for producing dental restorations, for example crowns and bridges.

WO 2017/155692 A1 (3M) claims a prefabricated dental crown characterized by its shape and its chemical composition. The composition comprises 20% to 70% by weight of nanofillers and 20% to 75% by weight of resin matrix which contain urethane (meth)acrylates and (meth)acrylates without a urethane unit.

WO 2015/126862 A1 (3M) discloses a dental composition comprising a particular asymmetric polymerizable monomer, initiator(s) and filler(s) in an amount of more than 20% by weight. The composition is suitable for producing filling material, crowns, bridges, inlays, onlays, veneers and milling blanks.

WO 2015/126666 A1 (3M) describes a dental composition comprising a particular asymmetric polymerizable monomer, initiator and filler component(s) in an amount of more than 20% by weight. The composition is suitable for producing filling material, dental cement, crowns, bridges, inlays, onlays, veneers, orthodontic devices and milling blanks.

WO 2018/231583 A1 (3M) claims a composition for producing dental crowns having a viscosity of less than 150 Pats and a shear rate of 1 s-1. The claimed composition comprises polymerizable (meth)acrylate(s) without a urethane component, polymerizable urethane (meth)acrylate(s), a filler matrix in an amount of 5% to 45% by weight with nanocluster(s) and optionally pyrogenic silica in an amount of less than 8% by weight, an initiator system comprising photoinitiator(s) and organic dye(s). The claimed composition further contains no plasticizer in an amount of more than 5% by weight.

EP 3 795 359 A1 (Shofu) discloses a composition for producing a dental restoration by 3D printing. The composition comprises a polymerizable monomer based on (meth)acrylate consisting of a polymerizable monomer based on (meth)acrylate which contains a urethane structure and a polymerizable monomer based on (meth)acrylate which contains no urethane structure, a cohesive inorganic filler and a photoinitiator in certain ratios to one another.

EP 1 508 834 A1 (3D Systems) claims a composition suitable for producing three-dimensional articles by stereolithography. This composition comprises at least one free-radically polymerizing organic substance, at least one initiator for free-radical polymerization, at least one filler comprising nanoparticles of the silicon dioxide type, at least one cationically polymerizable organic substance, at least one cationic polymerization initiator, optionally at least one hydroxyl-functional compound and optionally at least one type of microparticle filler.

WO 2014/098956 A1 (Dentca) describes a composition for producing artificial teeth and prostheses which comprises difunctional bisphenol-A dimethacrylate, multifunctional methacrylate, urethane dimethacrylate, surface-modified fine particles based on silicon dioxide, a photopolymerization initiator, a colorant and at least one type of stabilizer.

WO 2001/012679 A1 (Deltamed) claims a composition containing the following constituents: 2% to 99% by weight of at least one compound containing at least one acrylate group and/or at least one methacrylate group and/or at least one vinyl group and/or at least one epoxy group and/or at least one oxetane group and/or at least one acryloyl-epoxy oligomer group and/or at least one methacryloyl-epoxy oligomer group and/or at least one resin composition based on at least one polymerizable polysiloxane, 0.01% to 7% by weight of at least one initiator, 0% to 5% by weight of at least one co-initiator, 0% to 85% by weight of one or more modifiers, such as fillers, dyes, pigments, flow improvers, thixotropic agents, polymeric thickeners, oxidizing additives, stabilizers and retarders. This composition is inter alia suitable for producing inlays, onlays, dental fillings, attachments, crowns, bridges, artificial teeth, pivot teeth, tooth prostheses and dental implants.

EP 3 669 854 A1 (GC) discloses a composition suitable inter alia for producing inlays, crowns, bridges and prostheses. The composition comprises a (meth)acrylate compound, a filler, a photopolymerization initiator and a phthalic acid derivative and/or a thiophene derivative, wherein the content of the phthalic acid derivative and/or the thiophene derivative is not less than 0.05% by weight and not more than 0.25% by weight.

The composition (with dynamic viscosity) of WO 2020/177921 A1 (Mühlbauer) contains free-radically photopolymerizable monomers and/or oligomers, an organically surface-modified and optionally partially agglomerated and/or aggregated nanoscale filler incorporated into the composition, at least one photoinitiator, optionally a stabilizer, optionally pigment particles and optionally stabilized radical for stereolithographic production of a dental shaped article, in particular bridges and crowns.

EP 3 479 171 A1 (3M) describes a process for producing a three-dimensional article. The process comprises providing a printable composition comprising a high-viscosity polymerizable component, a temporary solvent and an initiator, selectively curing the printable composition to form an article which is in the shape of the three-dimensional object, removing a substantial amount of the temporary solvent from the article and optionally curing the unpolymerized polymerizable component that remains before or after the removal of a substantial amount of the temporary solvent.

WO 2020/016282 A1 (Fraunhofer-Gesellschaft zur Förderung der angewandten Forschung) claims a process for producing a shaped article using a radiation-induced printing process and a shaped article based on an organically polymerized silicic acid (hetero)polycondensate produced by organic polymerization of a polysiloxane component. The shaped article features an elastic modulus of at least 1.4 GPa and is suitable for use as a temporary or permanent replacement tooth.

Although the prior art discloses a number of compositions employable for 3D printing of dental restorations there is in the context of the advancing field of digital dentistry an increasing need for materials whose mechanical properties are comparable to the conventional materials. Particularly with regard to sedimentation stability, there is a considerable need for improvement. The hitherto disclosed printing resins generally require shaking before use. However, with increasing usage time in the resin bath of the 3D printer renewed sedimentation takes place and product quality decreases or the printing resin must be discarded and replaced.

It is accordingly an object of the invention to provide compositions which have good mechanical properties such as high strength and high abrasion stability. The compositions must also have a low viscosity to allow their reliable use in 3D printing processes (SLA, DLP) and therein ensure additive manufacturing of dental restorations with high precision. The compositions shall further have a high degree of sedimentation stability to allow production of uniformly good products during the entirety of the 3D printing process.

In one aspect, the present disclosure is directed to a free-radically polymerizable composition for 3D printing of dental crowns, bridges, prosthetic teeth or full prostheses comprising:
a. a total amount of free-radically polymerizable (meth)acrylic compounds in an amount of 50% to 89% by weight,
b. a total amount of inorganic fillers in an amount of 10% to 49% by weight, and
c. a total amount of photoinitiators in an amount of 0.1% to 5% by weight, in each case based on the total mass of the polymerizable composition, wherein the total amount (B) of inorganic fillers comprises:
   i. a first sub-amount (B1) of dental glass, and
   ii. a second sub-amount (B2) of pyrogenic silica, and wherein the mass ratio of B1 to B2 is in the range from 10: 1 to 1:1.

In another aspect, the present disclosure is directed to a dental crown, bridge, prosthetic tooth or full prosthesis produced by 3D printing of a free-radically polymerizable composition as described herein.

In another aspect, the present disclosure is directed to the free-radically polymerizable composition as described herein for use in a therapeutic method, preferably in a therapeutic method as a temporary crown or bridge, permanent crown or bridge, prosthetic tooth, prosthesis base, partial prosthesis or full prosthesis.

### DETAILED DESCRIPTION OF THE DISCLOSURE

The present disclosure relates to free-radically polymerizable compositions for 3D printing of dental crowns, bridges, prosthetic teeth or full prostheses.

In one aspect, it is an object of the disclosure to provide compositions which have good mechanical properties such as high strength and high abrasion stability. The compositions must also have a low viscosity to allow their reliable use in 3D printing processes (SLA, DLP) and therein ensure additive manufacturing of dental restorations with high precision. The compositions shall further have a high degree of sedimentation stability to allow production of uniformly good products during the entirety of the 3D printing process.

This object is achieved by a free-radically polymerizable composition for 3D printing of dental crowns, bridges, prosthetic teeth or full prostheses comprising:
a. a total amount of free-radically polymerizable (meth)acrylic compounds (“A”) in an amount of 50% to 89% by weight,
b. a total amount of inorganic fillers (“B”) in an amount of 10% to 49% by weight and,
c. a total amount of photoinitiators (“C”) in an amount of 0.1% to 5% by weight, in each case based on the total mass of the polymerizable composition, characterized in that the total amount (B) of inorganic fillers comprises:
   i. a first sub-amount (B1) of dental glass, and
   ii. a second sub-amount (B2) of pyrogenic silica, and wherein the mass ratio of B1 to B2 is in the range from 10:1 to 1:1.

In a preferred embodiment the mass ratio of B1 to B2 is in the range from 5:1 to 1.5:1, preferably in the range from 3.5:1 to 2:1.

In a preferred embodiment the free-radically polymerizable composition for 3D printing of dental crowns, bridges, prosthetic teeth or full prostheses comprises:
a. in an amount of 56% to 85% by weight, preferably 67% to 84% by weight,
b. in an amount of 10% to 40% by weight, preferably 15% to 30% by weight, and
c. in an amount of 0.1% to 4% by weight, preferably 0.5% to 3% by weight, in each case based on the total mass of the polymerizable composition.

Preference is given to a free-radically polymerizable composition for 3D printing of dental crowns, bridges, prosthetic teeth or full prostheses, wherein the dental glass B1 has:
a. an average particle size in the range from 0.4 to 5 um, preferably 0.4 to 2 um, particularly preferably in the range from 0.4 to 1.0 um, and/or (preferably “and”)
b. is selected from the group consisting of barium silicate glasses, barium aluminum silicate glasses, barium borosilicate glasses, barium boroaluminum silicate glasses, barium borofluoroaluminum silicate glasses, strontium silicate glasses, strontium aluminum silicate glasses, strontium borosilicate glasses, strontium boroaluminum silicate glasses, strontium borofluoroaluminum silicate glasses and zirconium silicate glasses, preferably selected from the group consisting of barium aluminum silicate glasses, barium borosilicate glasses and barium boroaluminum silicate glasses, and/or (preferably “and”)
c. is organically surface-modified, preferably organically surface-modified with 3-methacryloxypropyl(trimethoxy)silane.

Preference is given to a free-radically polymerizable composition for 3D printing of dental crowns, bridges, prosthetic teeth or full prostheses, wherein the total amount (A) of polymerizable (meth)acrylic compounds comprises:
a. a first sub-amount (A1) of difunctional, aromatic (meth)acrylates,
b. a second sub-amount (A2) of difunctional (meth)acrylates which comprise one or more urethane groups, and
c. a third sub-amount (A3) of monofunctional (meth)acrylates.

In a particularly preferred embodiment, the free-radically polymerizable composition for 3D printing of dental crowns, bridges, prosthetic teeth or full prostheses comprises:
a. (A1) in an amount of 20% to 60% by weight, preferably 30% to 50% by weight,
b. (A2) in an amount of 10% to 40% by weight, preferably 15% to 30% by weight, and
c. (A3) in an amount of 1% to 10% by weight, preferably 2% to 8% by weight, and/or (preferably “and”)
d. (B1) in an amount of 5% to 44.5% by weight, preferably 6.7% to 33.3% by weight, particularly preferably 10.7% to 23.3% by weight, and
e. (B2) in an amount of 0.9% to 24.5% by weight, preferably 1.7% to 13.3% by weight, particularly preferably 3.3% to 8.6% by weight, in each case based on the total mass of the polymerizable composition.

In a preferred embodiment A1 is selected from the group consisting of 2,2-bis[4-(meth)acryloyloxyethoxyphenyl]propane, 2,2-bis[4-(meth)acryloyloxydiethoxyphenyl]propane, 2,2-bis[4-(meth)acryloyloxytriethoxyphenyl]propane, 2,2-bis[4-(meth)acryloyloxytetraethoxyphenyl]propane, 2,2-bis[4-(meth)acryloyloxypentaethoxyphenyl]propane, 2,2-bis[4-(meth)acryloyloxydipropoxyphenyl]propane, 2,2-bis[4-(meth)acryloyloxyethoxyphenyl]-2-[4-(meth)acryloyloxydiethoxyphenyl]propane, 2-[4-(meth)acryloyloxydiethoxyphenyl]-2-[4-(meth)acryloyloxytriethoxyphenyl]propane, 2-[4-(meth)acryloyloxydipropoxyphenyl]-2-[4-(meth)acryloyloxytriethoxyphenyl]propane, 2,2-bis[4-(meth)acryloyloxyisopropoxyphenyl]propane, ethoxylated bisphenol A di(meth)acrylate and propoxylated bisphenol A di(meth)acrylate, and/or (preferably “and”)

A2 is selected from the group consisting of 3,14-dioxa-4,13-dioxo-5,12-diazahexadecane 1,16-dioxydi(meth)acrylate, 7,7,9-trimethyl-3,14-dioxa-4,13-dioxo-5, 12-diazahexadecane 1,16-dioxydi(meth)acrylate, 7,9,9-trimethyl-3,14-dioxa-4,13-dioxo-5,12-diazahexadecane 1,16-dioxydi(meth)acrylate and 1,5,5-trimethyl-1-[(2-(meth)acryloyloxyethyl)carbamoylmethyl]-3-(2-(meth)acryloyloxyethyl)carbamoylcyclohexane (CAS 42405-01-6 methacrylate; CAS 42404-50-2 acrylate), and/or (preferably “and”)

A3 is selected from the group consisting of 2-hydroxyethyl (meth)acrylate, 2-hydroxypropyl (meth)acrylate, 3-hydroxypropyl (meth)acrylate, tetrahydrofurfuryl (meth)acrylate, 4-(meth)acryloylmorpholine, cyclohexyl (meth)acrylate, 3,3,5-trimethylcyclohexyl (meth)acrylate, 4-(1,1-dimethylethyl)cyclohexyl (meth)acrylate, isobornyl (meth)acrylate, (octahydro-4,7-methano-1H-indenyl)methyl (meth)acrylate, benzyl (meth)acrylate, 2-phenoxyethyl (meth)acrylate, 2-(2-phenoxyethoxy)ethyl (meth)acrylate, 2-[2-(2-phenoxyethoxy)ethoxy]ethyl (meth)acrylate, ethoxylated 2-phenoxyethyl (meth)acrylate, 2-ethoxyethyl (meth)acrylate, 2-(2-ethoxyethoxy)ethyl (meth)acrylate, 2-[2-(2-ethoxyethoxy)ethoxy]ethyl (meth)acrylate, 2-methoxyethyl (meth)acrylate, 2-(2-methoxyethoxy)ethyl (meth)acrylate, 2-[2-(2-methoxyethoxy)ethoxy]ethyl (meth)acrylate, hexyl (meth)acrylate, 2-ethylhexyl (meth)acrylate, decyl (meth)acrylate, isodecyl (meth)acrylate, dodecyl (meth)acrylate, 2-hydroxy-3-(prop-2-enoyloxy)propyl 2-methyl-2-propylhexanoate and (5-ethyl-1,3-dioxan-5-yl)methyl (meth)acrylate.

In one aspect, a composition according to the invention comprises 50% to 89% by weight, preferably 56% to 85% by weight, particularly preferably 67% to 84% by weight, of a total amount (A) of free-radically polymerizable (meth)acrylic compounds, in each case based on the total mass of the polymerizable composition.

In the context of the present disclosure, the term (meth)acrylic compounds are to be understood as meaning both acrylic compounds and methacrylic compounds.

In a preferred embodiment, the total amount (A) of polymerizable (meth)acrylic compounds comprises:
a. a first sub-amount (A1) of difunctional, aromatic (meth)acrylates,
b. a second sub-amount (A2) of difunctional (meth)acrylates comprising one or more urethane groups and
c. a third sub-amount (A3) of monofunctional (meth)acrylates.

In a preferred embodiment the total amount (A) of polymerizable (meth)acrylic compounds comprises:
a. (A1) in an amount of 20% to 60% by weight, preferably 30% to 50% by weight,
b. (A2) in an amount of 10% to 40% by weight, preferably 15% to 30% by weight,
c. (A3) in an amount of 1% to 10% by weight, preferably 2% to 8% by weight, in each case based on the total mass of the polymerizable composition.

Furthermore, a composition according to the invention may also comprise in the total amount (A) a fourth sub-amount (A4) of polymerizable (meth)acrylic compounds which is distinct from A1, A2 or A3.

It is preferable when the total amount (A) of polymerizable (meth)acrylic compounds of such a composition comprises: (A1) in an amount of 20% to 60% by weight, preferably 30% to 50% by weight:
a. (A2) in an amount of 10% to 40% by weight, preferably 15% to 30% by weight,
b. (A3) in an amount of 1% to 10% by weight, preferably 2% to 8% by weight, and
c. (A4) in an amount of 0% to 20% by weight, preferably 1% to 8% by weight, in each case based on the total mass of the polymerizable composition.

It is preferable when the sub-amount A1 comprises one or more (meth)acrylic compounds selected from the group consisting of 2,2-bis[4-(meth)acryloyloxyethoxyphenyl]propane, 2,2-bis[4-(meth)acryloyloxydiethoxyphenyl]propane, 2,2-bis[4-(meth)acryloyloxytriethoxyphenyl]propane, 2,2-bis[4-(meth)acryloyloxytetraethoxyphenyl]propane, 2,2-bis[4-(meth)acryloyloxypentaethoxyphenyl]propane, 2,2-bis[4-(meth)acryloyloxydipropoxyphenyl]propane, 2,2-bis[4-(meth)acryloyloxyethoxyphenyl]-2-[4-(meth)acryloyloxydiethoxyphenyl]propane, 2-[4-(meth)acryloyloxydiethoxyphenyl]-2-[4-(meth)acryloyloxytriethoxyphenyl]propane, 2-[4-(meth)acryloyloxydipropoxyphenyl]-2-[4-(meth)acryloyloxytriethoxyphenyl]propane, 2,2-bis[4-(meth)acryloyloxyisopropoxyphenyl]propane, ethoxylated bisphenol A di(meth)acrylate and propoxylated bisphenol A di(meth)acrylate.

It is preferable when the sub-amount A1 comprises 2,2-bis[4-(2-hydroxy-3-(meth)acryloyloxypropoxy)phenyl]propane (bis-GMA) in an amount of less than 8% by weight, preferably less than 4% by weight, particularly preferably less than 2% by weight, in each case based on the total mass of the polymerizable composition.

In a preferred embodiment, a composition according to the invention comprises substantially no 2,2-bis[4-(2-hydroxy-3-(meth)acryloyloxypropoxy)phenyl]propane or absolutely no 2,2-bis[4-(2-hydroxy-3-(meth)acryloyloxypropoxy)phenyl]propane.

It is preferable when the sub-amount A2 comprises one or more (meth)acrylic compounds selected from the group consisting of 3,14-dioxa-4,13-dioxo-5,12-diazahexadecane 1,16-dioxydi(meth)acrylate, 7,7,9-trimethyl-3,14-dioxa-4, 13-dioxo-5, 12-diazahexadecane 1,16-dioxydi(meth)acrylate, 7,9,9-trimethyl-3,14-dioxa-4,13-dioxo-5,12-diazahexadecane 1,16-dioxydi(meth)acrylate and 1,5,5-trimethyl-1-[(2-(meth)acryloyloxyethyl)carbamoylmethyl]-3-(2-(meth)acryloyloxyethyl)carbamoylcyclohexane (CAS 42405-01-6 methacrylate; CAS 42404-50-2 acrylate).

It is preferable when the sub-amount A3 comprises one or more (meth)acrylic compounds selected from the group consisting of 2-hydroxyethyl (meth)acrylate, 2-hydroxypropyl (meth)acrylate, 3-hydroxypropyl (meth)acrylate, tetrahydrofurfuryl (meth)acrylate, 4-(meth)acryloylmorpholine, cyclohexyl (meth)acrylate, 3,3,5-trimethylcyclohexyl (meth)acrylate, 4-(1,1-dimethylethyl)cyclohexyl (meth)acrylate, isobornyl (meth)acrylate, (octahydro-4,7-methano-1H-indenyl)methyl (meth)acrylate, benzyl (meth)acrylate, 2-phenoxyethyl (meth)acrylate, 2-(2-phenoxyethoxy)ethyl (meth)acrylate, 2-[2-(2-phenoxyethoxy)ethoxy]ethyl (meth)acrylate, ethoxylated 2-penoxyethyl (meth)acrylate, 2-ethoxyethyl (meth)acrylate, 2-(2-ethoxyethoxy)ethyl (meth)acrylate, 2-[2-(2-ethoxyethoxy)ethoxy]ethyl (meth)acrylate, 2-methoxyethyl (meth)acrylate, 2-(2-methoxyethoxy)ethyl (meth)acrylate, 2-[2-(2-methoxyethoxy)ethoxy]ethyl (meth)acrylate, hexyl (meth)acrylate, 2-ethylhexyl (meth)acrylate, decyl (meth)acrylate, isodecyl (meth)acrylate, dodecyl (meth)acrylate, 2-hydroxy-3-(prop-2-enoyloxy)propyl 2-methyl-2-propylhexanoate and (5-ethyl-1,3-dioxan-5-yl)methyl (meth)acrylate.

It is preferable when the sub-amount A4 comprises one or more (meth)acrylic compounds selected from the group consisting of ethylene glycol di(meth)acrylate, diethylene glycol di(meth)acrylate, triethylene glycol di(meth)acrylate, tetraethylene glycol di(meth)acrylate, polyethylene glycol di(meth)acrylate, propylene glycol di(meth)acrylate, dipropylene glycol di(meth)acrylate, tripropylene glycol di(meth)acrylate, tetrapropylene glycol di(meth)acrylate, polypropylene glycol di(meth)acrylate, 1,4-butanediol di(meth)acrylate, 1,3-butanediol di(meth)acrylate, 1,6-hexanediol di(meth)acrylate, 1,10-decanediol di(meth)acrylate, 1,12-dodecanediol di(meth)acrylate, 2-hydroxypropyl 1,3-di(meth)acrylate, 3-hydroxypropyl 1,2-di(meth)acrylate, neopentyl glycol di(meth)acrylate and 3(4),8(9)-bis((meth)acryloyloxymethyl)tricyclo[5.2.1.02.6]decane.

In one aspect, a composition according to the invention comprises 10% to 49% by weight, preferably 10% to 40% by weight, particularly preferably 15% to 30% by weight, of a total amount (B) of inorganic fillers, in each case based on the total mass of the polymerizable composition.

In one aspect, the total amount (B) of inorganic fillers comprises a first sub-amount (B1) of dental glass and a second sub-amount (B2) of pyrogenic silica, wherein the mass ratio of B1 to B2 is in the range from 10:1 to 1:1, preferably in the range from 5:1 to 1.5:1, particularly preferably in the range from 3.5:1 to 2:1.

Dental glass is to be understood as meaning glasses that are suitable for use in the dental sector. They feature inter alia a high purity and contain no toxic constituents (for example lead or arsenic). Such dental glasses are commercially available in various particle sizes inter alia from SCHOTT under the designations G018-307, G018-163, G018-093, GM39923, GM32087, G018-308, GM27884, GM31685, G018-053, 8235, G018-186, GM31684, G018-310, G018-159, G018-161, GM35429, G018-090 and G018-117 or from FERRO under the designations IS 50 1101, IS 50 1102 and IS 50 1103.

It is preferable when a composition according to the invention contains B1 in an amount of 5% to 44.5% by weight, preferably 6.7% to 33.3% by weight, particularly preferably 10.7% to 23.3% by weight, in each case based on the total mass of the polymerizable composition.

It is preferable when B1 is selected from the group consisting of barium silicate glasses, barium aluminum silicate glasses, barium borosilicate glasses, barium boroaluminum silicate glasses, barium borofluoroaluminum silicate glasses, strontium silicate glasses, strontium aluminum silicate glasses, strontium borosilicate glasses, strontium boroaluminum silicate glasses, strontium borofluoroaluminum silicate glasses and zirconium silicate glasses, preferably selected from the group consisting of barium aluminum silicate glasses, barium borosilicate glasses and barium boroaluminum silicate glasses.

In a preferred embodiment, B1 has an average particle size in the range from 0.4 to 5 um, preferably 0.4 to 2 um, particularly preferably in the range from 0.4 to 1.0 um.

The average particle size relates to d50 values determined by static light scattering with volume-weighted evaluation.

In a further preferred embodiment B1 is organically surface-modified, preferably organically surface-modified with 3-methacryloxypropyl(trimethoxy)silane.

It is preferable when a composition according to the invention contains B2 in an amount of 0.9% to 24.5% by weight, preferably 1.7% to 13.3% by weight, particularly preferably 3.3% to 8.6% by weight, in each case based on the total mass of the polymerizable composition.

In a preferred embodiment the pyrogenic silica B2 has a primary particle size in the range from 5 to 25 nm, preferably in the range from 10 to 20 nm.

The primary particle sizes of B2 are determinable by transmission electron microscopy (TEM). Corresponding processes are described in “Untersuchungen zur fließregulierenden Eigenschaft hochdisperser Fällungskieselsäuren” (Anne-Kathrin Müller, Thesis, Würzburg 2008).

In a further preferred embodiment, the pyrogenic silica B2 is organically surface-modified, preferably organically surface-modified with 3-methacryloxypropyl(trimethoxy)silane, hexamethyldisilazane or dichloro(dimethyl)silane.

In a further preferred embodiment, the pyrogenic silica B2 has a BET surface area in the range from 100 to 300 m2/g, preferably in the range from 120 to 200 m2/g. The BET surface area is preferably determined according to DIN ISO 9277:2014.

In a further preferred embodiment, the pyrogenic silica B2 has a tamped density in the range from 100 to 300 g/L, preferably in the range from 120 to 250 g/L. The tamped density is preferably determined according to DIN ISO 787-11:1995.

In a further preferred embodiment, the pyrogenic silica B2 is structure-modified and/or compressed and post-milled, preferably structure-modified by compressing and post-milling.

Structure modification is to be understood as meaning a process for structure alteration (generally for destructuring) of pyrogenic metal oxides, in particular pyrogenic silicas, wherein the microscopic 3D network of the aggregates and the agglomerates is altered so as to form materials having new properties. These thus feature especially a reduced thickening activity and increased tamped density. A suitable process for structure modification of pyrogenic silicas is described in DE 196 16 781 A1 (Degussa AG).

In one aspect, a composition according to the invention contains 0.1% to 5% by weight, preferably 0.1% to 4% by weight, particularly preferably 0.5% to 3% by weight, in each case based on the total mass of the polymerizable composition, of a total amount of photoinitiators (C).

Employable photoinitiators include the suitable compounds customary for (meth)acrylic systems. It is advantageous to use alpha-diketones, benzoin alkyl ethers, thioxanthones, benzophenones, acetophenones, acylphosphine oxides or acylgermanium compounds. It is preferable to use monoacylphosphine oxides or bisacylphosphine oxides.

In a preferred embodiment, (C) is selected from the group consisting of alpha-diketones, benzoin alkyl ethers, thioxanthones, benzophenones, acetophenones, acylphosphine oxides and acylgermanium compounds, preferably selected from the group consisting of 1-hydroxycyclohexylbenzophenone, 4-(2-hydroxyethoxy)phenyl-(2-hydroxy-2-propyl)ketone, 2-hydroxy-2-methylpropiophenone, 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate (CAS 84434-11-7) and (poly(oxy-1,2-ethanediyl),′,a″a″-1,2,3-propanetriyltris[@-[phenyl(2,4,6-trimethylbenzoyl)phosphinyl]oxy] polymer) (CAS 1834525-17-5), particularly preferably selected from the group consisting of 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate and (poly(oxy-1,2-ethanediyl),′,a″a″-1,2,3-propanetriyltris[@-[phenyl(2,4,6-trimethylbenzoyl)phosphinyl]oxy] polymer).

In a preferred embodiment, a composition according to the invention additionally comprises 0.001% to 1% by weight, preferably 0.01% to 0.5% by weight, particularly preferably 0.01% to 0.3% by weight, in each case based on the total mass of the polymerizable composition, of one or more stabilizers (D).

It is preferable when (D) is selected from the group consisting of hydroquinone, hydroquinone monomethyl ether, 2,6-di-tert-butyl-4-methylphenol, tert-butylhydroxyanisole and 2,2,6,6-tetramethylpiperidine-1-oxyl.

In order to adjust the colour of the material for the desired application a composition according to the invention may additionally contain one or more colorants (E). Employable colorants include inorganic colour pigments and organic colour pigments or dyes. For tooth-coloured applications, white, yellow, orange, red, brown and/or black colorants are employed.

It is preferable when (E) comprises titanium dioxide and/or iron oxide.

In a preferred embodiment, a composition according to the invention contains 0.0001% to 1% by weight, preferably 0.0001% to 0.5% by weight, particularly preferably 0.0001% to 0.1% by weight, in each case based on the total mass of the polymerizable composition, of one or more colorants (E).

In a particularly preferred embodiment, the viscosity of the free-radically polymerizable composition for 3D printing of dental crowns, bridges, prosthetic teeth or full prostheses is:
a. at a shear rate of 0.01 s⁻¹ in the range from 100 to 500 Pats, preferably in the range from 200 to 270 Pa*s, and/or (preferably “and”)
b. at a shear rate of 0.1 s⁻¹ in the range from 10 to 50 Pa*s, preferably in the range from 30 to 40 Pa*s, and/or (preferably “and”)
c. at a shear rate of 1 s⁻¹ in the range from 2 to 20 Pa*s, preferably in the range from 6.5 to 10.5 Pa*s, and/or (preferably “and”)
d. at a shear rate of 10 s⁻¹ in the range from 1 to 10 Pa*s, preferably in the range from 2.5 to 6.0 Pa*s.

The loss factor tan 8 is the quotient of the loss modulus G″ (imaginary portion) and the storage modulus G′ (real portion). In rheology it describes the viscoelastic behaviour. The lower the loss factor, the more the behaviour of a specimen corresponds to that of an ideally elastic solid. The higher the loss factor, the more the behaviour of a specimen approximates that of an ideally viscous liquid with Newtonian flow behaviour. At tan 8=1 a transition from a gel to a liquid takes place. It is therefore preferable when at low shear stress (small deflections up to 0.01%), as occurs in the resting state for example during storage in the bottle or resin bath, compositions according to the invention have a tan 8 of less than 1. They then exhibit a gel character which results in good sedimentation stability. It is additionally preferable when at higher shear stress (larger deflections above 0.1%), as occurs in particular during the 3D printing operation in the region of the moving build platform, compositions according to the invention have a tan 8 of more than 1. They then behave like liquids, can exhibit good, continued flow and ensure high precision in 3D printing.

Preference is therefore given to a free-radically polymerizable composition for 3D printing of dental crowns, bridges, prosthetic teeth or full prostheses, wherein the quotient tan 8 of the loss modulus G″ and the storage modulus G′ under oscillating shear stress:
a. for deflections in the range from 0.001% to 0.01% is less than 1, preferably in the range from 0.4 to 0.99, particularly preferably in the range from 0.6 to 0.95, and
b. for deflections in the range from 1% to 10% is greater than 1, preferably in the range from 1.01 to 5, particularly preferably in the range from 1.05 to 3.

The present invention also provides a dental crown, bridge, prosthetic tooth or full prosthesis that has been produced by 3D printing of a free-radically polymerizable composition according to the invention.

The present invention also provides a free-radically polymerizable composition for use in a therapeutic method, preferably in a therapeutic method as a:
a. temporary crown or bridge,
b. permanent crown or bridge,
c. prosthetic tooth,
d. prosthesis base,
e. partial prosthesis or
f. full prosthesis.

### Embodiments

Free-radically polymerizable composition for 3D printing of dental crowns, bridges, prosthetic teeth or full prostheses comprising:
a. a total amount of free-radically polymerizable (meth)acrylic compounds in an amount of 50% to 89% by weight,
b. a total amount of inorganic fillers in an amount of 10% to 49% by weight and
c. a total amount of photoinitiators in an amount of 0.1% to 5% by weight, in each case based on the total mass of the polymerizable composition, characterized in that the total amount (B) of inorganic fillers comprises:
   i. a first sub-amount (B1) of dental glass, and
   ii. a second sub-amount (B2) of pyrogenic silica
   iii. and wherein the mass ratio of B1 to B2 is in the range from 10:1 to 1:1.

Free-radically polymerizable composition for 3D printing of dental crowns, bridges, prosthetic teeth or full prostheses according to aspect 1, wherein the mass ratio of B1 to B2 is in the range from 5:1 to 1.5:1, preferably in the range from 3.5:1 to 2:1.

Free-radically polymerizable composition for 3D printing of dental crowns, bridges, prosthetic teeth or full prostheses according to either of the preceding aspects, comprising:
a. in an amount of 56% to 85% by weight, preferably 67% to 84% by weight,
b. in an amount of 10% to 40% by weight, preferably 15% to 30% by weight and
c. in an amount of 0.1% to 4% by weight, preferably 0.5% to 3% by weight,
d. in each case based on the total mass of the polymerizable composition.

Free-radically polymerizable composition for 3D printing of dental crowns, bridges, prosthetic teeth or full prostheses according to any of the preceding aspects, wherein the dental glass B1 has an average particle size in the range from 0.4 to 5 μm, preferably 0.4 to 2 μm, particularly preferably in the range from 0.4 to 1.0 μm.

Free-radically polymerizable composition for 3D printing of dental crowns, bridges, prosthetic teeth or full prostheses according to any of the preceding aspects, wherein the average particle size of B1 is determined by static light scattering as the d50 value with volume-weighted evaluation.

Free-radically polymerizable composition for 3D printing of dental crowns, bridges, prosthetic teeth or full prostheses according to any of the preceding aspects, wherein the dental glass B1 is selected from the group consisting of barium silicate glasses, barium aluminum silicate glasses, barium borosilicate glasses, barium boroaluminum silicate glasses, barium borofluoroaluminum silicate glasses, strontium silicate glasses, strontium aluminum silicate glasses, strontium borosilicate glasses, strontium boroaluminum silicate glasses, strontium borofluoroaluminum silicate glasses and zirconium silicate glasses, preferably selected from the group consisting of barium aluminum silicate glasses, barium borosilicate glasses and barium boroaluminum silicate glasses.

Free-radically polymerizable composition for 3D printing of dental crowns, bridges, prosthetic teeth or full prostheses according to any of the preceding aspects, wherein the dental glass B1 is organically surface-modified, preferably organically surface-modified with 3-methacryloxypropyl(trimethoxy)silane.

Free-radically polymerizable composition for 3D printing of dental crowns, bridges, prosthetic teeth or full prostheses according to any of the preceding aspects, wherein the pyrogenic silica B2 has a primary particle size in the range from 5 to 25 nm, preferably in the range from 10 to 20 nm.

Free-radically polymerizable composition for 3D printing of dental crowns, bridges, prosthetic teeth or full prostheses according to any of the preceding aspects, wherein the pyrogenic silica B2 has a primary particle size in the range from 5 to 25 nm, preferably in the range from 10 to 20 nm, wherein the primary particle size is determined as a volume-weighted distribution by transmission electron microscopy.

Free-radically polymerizable composition for 3D printing of dental crowns, bridges, prosthetic teeth or full prostheses according to any of the preceding aspects, wherein the pyrogenic silica B2 is organically surface-modified, preferably organically surface-modified with 3-methacryloxypropyl(trimethoxy)silane, hexamethyldisilazane or dichloro(dimethyl)silane.

Free-radically polymerizable composition for 3D printing of dental crowns, bridges, prosthetic teeth or full prostheses according to any of the preceding aspects, wherein the pyrogenic silica B2 has a BET surface area in the range from 100 to 300 m2/g, preferably in the range from 120 to 200 m2/g.

Free-radically polymerizable composition for 3D printing of dental crowns, bridges, prosthetic teeth or full prostheses according to any of the preceding aspects, wherein the pyrogenic silica B2 has a BET surface area in the range from 100 to 300 m2/g, preferably in the range from 120 to 200 m2/g, wherein the BET surface area is determined according to DIN ISO 9277:2014.

Free-radically polymerizable composition for 3D printing of dental crowns, bridges, prosthetic teeth or full prostheses according to any of the preceding aspects, wherein the pyrogenic silica B2 has a tamped density in the range from 100 to 300 g/L, preferably in the range from 120 to 250 g/L.

Free-radically polymerizable composition for 3D printing of dental crowns, bridges, prosthetic teeth or full prostheses according to any of the preceding aspects, wherein the pyrogenic silica B2 has a tamped density in the range from 100 to 300 g/L, preferably in the range from 120 to 250 g/L, wherein the tamped density is determined according to DIN ISO 787-11:1995.

Free-radically polymerizable composition for 3D printing of dental crowns, bridges, prosthetic teeth or full prostheses according to any of the preceding aspects, wherein the pyrogenic silica B2 is structure-modified and/or compressed and post-milled, preferably structure-modified by compressing and post-milling.

Free-radically polymerizable composition for 3D printing of dental crowns, bridges, prosthetic teeth or full prostheses according to any of the preceding aspects, wherein the total amount (A) of polymerizable (meth)acrylic compounds comprises:
a. a first sub-amount (A1) of difunctional, aromatic (meth)acrylates,
b. a second sub-amount (A2) of difunctional (meth)acrylates which comprise one or more urethane groups and
c. a third sub-amount (A3) of monofunctional (meth)acrylates.

Free-radically polymerizable composition for 3D printing of dental crowns, bridges, prosthetic teeth or full prostheses according to any of the preceding aspects, comprising:
a. (A1) in an amount of 20% to 60% by weight, preferably 30% to 50% by weight,
b. (A2) in an amount of 10% to 40% by weight, preferably 15% to 30% by weight, and
c. (A3) in an amount of 1% to 10% by weight, preferably 2% to 8% by weight, in each case based on the total mass of the polymerizable composition.

Free-radically polymerizable composition for 3D printing of dental crowns, bridges, prosthetic teeth or full prostheses according to any of the preceding aspects, wherein the total amount (A) of polymerizable (meth)acrylic compounds comprises a fourth sub-amount (A4) of polymerizable (meth)acrylic compounds which is distinct from A1, A2 or A3.

Free-radically polymerizable composition for 3D printing of dental crowns, bridges, prosthetic teeth or full prostheses according to any of the preceding aspects, comprising:
a. (A1) in an amount of 20% to 60% by weight, preferably 30% to 50% by weight,
b. (A2) in an amount of 10% to 40% by weight, preferably 15% to 30% by weight,
c. (A3) in an amount of 1% to 10% by weight, preferably 2% to 8% by weight and
d. (A4) in an amount of 0% to 20% by weight, preferably 1% to 8% by weight,
e. in each case based on the total mass of the polymerizable composition.

Free-radically polymerizable composition for 3D printing of dental crowns, bridges, prosthetic teeth or full prostheses according to any of the preceding aspects, comprising:
a. (B1) in an amount of 5% to 44.5% by weight, preferably 6.7% to 33.3% by weight, particularly preferably 10.7% to 23.3% by weight, and
b. (B2) in an amount of 0.9% to 24.5% by weight, preferably 1.7% to 13.3% by weight, particularly preferably 3.3% to 8.6% by weight,
c. in each case based on the total mass of the polymerizable composition.

Free-radically polymerizable composition for 3D printing of dental crowns, bridges, prosthetic teeth or full prostheses according to any of the preceding aspects, wherein A1 is selected from the group consisting of 2,2-bis[4-(meth)acryloyloxyethoxyphenyl]propane, 2,2-bis[4-(meth)acryloyloxydiethoxyphenyl]propane, 2,2-bis[4-(meth)acryloyloxytriethoxyphenyl]propane, 2,2-bis[4-(meth)acryloyloxytetraethoxyphenyl]propane, 2,2-bis[4-(meth)acryloyloxypentaethoxyphenyl]propane, 2,2-bis[4-(meth)acryloyloxydipropoxyphenyl]propane, 2,2-bis[4-(meth)acryloyloxyethoxyphenyl]-2-[4-(meth)acryloyloxydiethoxyphenyl]propane, 2-[4-(meth)acryloyloxydiethoxyphenyl]-2-[4-(meth)acryloyloxytriethoxyphenyl]propane, 2-[4-(meth)acryloyloxydipropoxyphenyl]-2-[4-(meth)acryloyloxytriethoxyphenyl]propane, 2,2-bis[4-(meth)acryloyloxyisopropoxyphenyl]propane, ethoxylated bisphenol A di(meth)acrylate and propoxylated bisphenol A di(meth)acrylate.

Free-radically polymerizable composition for 3D printing of dental crowns, bridges, prosthetic teeth or full prostheses according to any of the preceding aspects, wherein the sub-amount A1 comprises 2,2-bis[4-(2-hydroxy-3-(meth)acryloyloxypropoxy)phenyl]propane (bis-GMA) in an amount of less than 8% by weight, preferably less than 4% by weight, particularly preferably less than 2% by weight, in each case based on the total mass of the polymerizable composition.

Free-radically polymerizable composition for 3D printing of dental crowns, bridges, prosthetic teeth or full prostheses according to any of the preceding aspects, wherein the sub-amount A1 comprises substantially no 2,2-bis [4-(2-hydroxy-3-(meth)acryloyloxypropoxy)phenyl]propane or absolutely no 2,2-bis [4-(2-hydroxy-3-(meth)acryloyloxypropoxy)phenyl]propane.

Free-radically polymerizable composition for 3D printing of dental crowns, bridges, prosthetic teeth or full prostheses according to any of the preceding aspects, wherein A2 is selected from the group consisting of 3,14-dioxa-4,13-dioxo-5,12-diazahexadecane 1,16-dioxydi(meth)acrylate, 7,7,9-trimethyl-3,14-dioxa-4,13-dioxo-5, 12-diazahexadecane 1,16-dioxydi(meth)acrylate, 7,9,9-trimethyl-3,14-dioxa-4, 13-dioxo-5,12-diazahexadecane 1,16-dioxydi(meth)acrylate and 1,5,5-trimethyl-1-[(2-(meth)acryloyloxyethyl)carbamoylmethyl]-3-(2-(meth)acryloyloxyethyl)carbamoylcyclohexane (CAS 42405-01-6 methacrylate; CAS 42404-50-2 acrylate).

Free-radically polymerizable composition for 3D printing of dental crowns, bridges, prosthetic teeth or full prostheses according to any of the preceding aspects, wherein A3 is selected from the group consisting of 2-hydroxyethyl (meth)acrylate, 2-hydroxypropyl (meth)acrylate, 3-hydroxypropyl (meth)acrylate, tetrahydrofurfuryl (meth)acrylate, 4-(meth)acryloylmorpholine, cyclohexyl (meth)acrylate, 3,3,5-trimethylcyclohexyl (meth)acrylate, 4-(1,1-dimethylethyl)cyclohexyl (meth)acrylate, isobornyl (meth)acrylate, (octahydro-4,7-methano-1H-indenyl)methyl (meth)acrylate, benzyl (meth)acrylate, 2-phenoxyethyl (meth)acrylate, 2-(2-phenoxyethoxy)ethyl (meth)acrylate, 2-[2-(2-phenoxyethoxy)ethoxy]ethyl (meth)acrylate, ethoxylated 2-penoxyethyl (meth)acrylate, 2-ethoxyethyl (meth)acrylate, 2-(2-ethoxyethoxy)ethyl (meth)acrylate, 2-[2-(2-ethoxyethoxy)ethoxy]ethyl (meth)acrylate, 2-methoxyethyl (meth)acrylate, 2-(2-methoxyethoxy)ethyl (meth)acrylate, 2-[2-(2-methoxyethoxy)ethoxy]ethyl (meth)acrylate, hexyl (meth)acrylate, 2-ethylhexyl (meth)acrylate, decyl (meth)acrylate, isodecyl (meth)acrylate, dodecyl (meth)acrylate, 2-hydroxy-3-(prop-2-enoyloxy)propyl 2-methyl-2-propylhexanoate and (5-ethyl-1,3-dioxan-5-yl)methyl (meth)acrylate.

Free-radically polymerizable composition for 3D printing of dental crowns, bridges, prosthetic teeth or full prostheses according to any of the preceding aspects, wherein A3 comprises a monofunctional (meth)acrylic compound having an alicyclic radical.

Free-radically polymerizable composition for 3D printing of dental crowns, bridges, prosthetic teeth or full prostheses according to any of the preceding aspects, wherein A4 is selected from the group consisting of ethylene glycol di(meth)acrylate, diethylene glycol di(meth)acrylate, triethylene glycol di(meth)acrylate, tetraethylene glycol di(meth)acrylate, polyethylene glycol di(meth)acrylate, propylene glycol di(meth)acrylate, dipropylene glycol di(meth)acrylate, tripropylene glycol di(meth)acrylate, tetrapropylene glycol di(meth)acrylate, polypropylene glycol di(meth)acrylate, 1,4-butanediol di(meth)acrylate, 1,3-butanediol di(meth)acrylate, 1,6-hexanediol di(meth)acrylate, 1,10-decanediol di(meth)acrylate, 1,12-dodecanediol di(meth)acrylate, 2-hydroxypropyl 1,3-di(meth)acrylate, 3-hydroxypropyl 1,2-di(meth)acrylate, neopentyl glycol di(meth)acrylate and 3(4),8(9)-bis((meth)acryloyloxymethyl)tricyclo[5.2.1.02.6]decane.

Free-radically polymerizable composition for 3D printing of dental crowns, bridges, prosthetic teeth or full prostheses according to any of the preceding aspects, comprising a total amount of photoinitiators (C) in an amount of 0.1% to 5% by weight, preferably 0.1% to 4% by weight, particularly preferably 0.5% to 3% by weight, in each case based on the total mass of the polymerizable composition.

Free-radically polymerizable composition for 3D printing of dental crowns, bridges, prosthetic teeth or full prostheses according to any of the preceding aspects, wherein (C) is selected from the group consisting of alpha-diketones, benzoin alkyl ethers, thioxanthones, benzophenones, acetophenones, acylphosphine oxides and acylgermanium compounds, preferably selected from the group consisting of 1-hydroxycyclohexylbenzophenone, 4-(2-hydroxyethoxy)phenyl-(2-hydroxy-2-propyl)ketone, 2-hydroxy-2-methylpropiophenone, 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate (CAS 84434-11-7) and (poly(oxy-1,2-ethanediyl),a,a′,a″-1,2,3-propanetriyltris[@-[phenyl(2,4,6-trimethylbenzoyl)phosphinyl]oxy] polymer) (CAS 1834525-17-5), particularly preferably selected from the group consisting of 2,4,6-trimethylbenzoyldiphenylphosphine oxide, phenylbis(2,4,6-trimethylbenzoyl)phosphine oxide, ethyl phenyl(2,4,6-trimethylbenzoyl)phosphinate and (poly(oxy-1,2-ethanediyl), a,a′,a″-1,2,3-propanetriyltris[@-[phenyl(2,4,6-trimethylbenzoyl)phosphinyl]oxy] polymer).

Free-radically polymerizable composition for 3D printing of dental crowns, bridges, prosthetic teeth or full prostheses according to any of the preceding aspects, comprising a total amount of stabilizers (D) in an amount of 0.001% to 1% by weight, preferably 0.01% to 0.5% by weight, particularly preferably 0.01% to 0.3% by weight, in each case based on the total mass of the polymerizable composition.

Free-radically polymerizable composition for 3D printing of dental crowns, bridges, prosthetic teeth or full prostheses according to any of the preceding aspects, wherein (D) is selected from the group consisting of hydroquinone, hydroquinone monomethyl ether, 2,6-di-tert-butyl-4-methylphenol, tert-butylhydroxyanisole and 2,2,6,6-tetramethylpiperidine-1-oxyl.

Free-radically polymerizable composition for 3D printing of dental crowns, bridges, prosthetic teeth or full prostheses according to any of the preceding aspects, comprising a total amount of colorants (E) in an amount of 0.0001% to 1% by weight, preferably 0.0001% to 0.5% by weight, particularly preferably 0.0001% to 0.1% by weight, in each case based on the total mass of the polymerizable composition.

Free-radically polymerizable composition for 3D printing of dental crowns, bridges, prosthetic teeth or full prostheses according to any of the preceding aspects, wherein (E) comprises titanium dioxide and/or iron oxide.

Free-radically polymerizable composition for 3D printing of dental crowns, bridges, prosthetic teeth or full prostheses according to any of the preceding aspects, wherein the viscosity at a shear rate of 0.01 s⁻¹ is in the range from 100 to 500 Pas, preferably in the range from 200 to 270 Pa*s.

Free-radically polymerizable composition for 3D printing of dental crowns, bridges, prosthetic teeth or full prostheses according to any of the preceding aspects, wherein the viscosity at a shear rate of 0.1 s⁻¹ is in the range from 10 to 50 Pa*s, preferably in the range from 30 to 40 Pa*s.

Free-radically polymerizable composition for 3D printing of dental crowns, bridges, prosthetic teeth or full prostheses according to any of the preceding aspects, wherein the viscosity at a shear rate of 1 s⁻¹ is in the range from 2 to 20 Pa*s, preferably in the range from 6.5 to 10.5 Pa*s.

Free-radically polymerizable composition for 3D printing of dental crowns, bridges, prosthetic teeth or full prostheses according to any of the preceding aspects, wherein the viscosity at a shear rate of 10 s⁻¹ is in the range from 1 to 10 Pa*s, preferably in the range from 2.5 to 6.0 Pa*s.

Free-radically polymerizable composition for 3D printing of dental crowns, bridges, prosthetic teeth or full prostheses according to any of the preceding aspects, wherein the quotient tan 8 of the loss modulus G″ and the storage modulus G′ under oscillating shear stress:
a. for deflections in the range from 0.001% to 0.01% is less than 1, preferably in the range from 0.4 to 0.99, particularly preferably in the range from 0.6 to 0.95, and
b. for deflections in the range from 1% to 10% is greater than 1, preferably in the range from 1.01 to 5, particularly preferably in the range from 1.05 to 3.

Free-radically polymerizable composition for 3D printing of dental crowns, bridges, prosthetic teeth or full prostheses according to any of the preceding aspects, wherein the quotient tan 8 of the loss modulus G″ and the storage modulus G′ under oscillating shear stress:
a. for deflections≤0.01% is less than 1, preferably in the range from 0.4 to 0.99, particularly preferably in the range from 0.6 to 0.95, and
b. for deflections >1% is greater than 1, preferably in the range from 1.01 to 5, particularly preferably in the range from 1.05 to 3.

Dental crown or bridge produced by 3D printing of a free-radically polymerizable composition according to any of aspects 1 to 39.

Dental prosthetic tooth produced by 3D printing of a free-radically polymerizable composition according to any of aspects 1 to 39.

Dental prosthesis base, partial prosthesis or full prosthesis produced by 3D printing of a free-radically polymerizable composition according to any of aspects 1 to 39.

Free-radically polymerizable composition according to any of aspects 1 to 39 for use in a therapeutic method, preferably in a therapeutic method as a:
a. temporary crown or bridge,
b. permanent crown or bridge,
c. prosthetic tooth,
d. prosthesis base,
e. partial prosthesis or
f. full prosthesis.

### Examples

The objectives and advantages of this disclosure are further elucidated by the following examples but the specific materials and the amounts thereof that are recited in these examples as well as other conditions and details should not be interpreted as unduly limiting this disclosure:
a. Bis-EMA-4: ethoxylated bisphenol A dimethacrylate (4 EO units; CAS 41637-38-1)
b. TMDI-UDMA: 7,7,9-trimethyl-3, 14-dioxa-4, 13-dioxo-5,12-diazahexadecane 1,16-dioxydi(meth)acrylate /7,9,9-trimethyl-3,14-dioxa-4,13-dioxo-5,12-diazahexadecane 1,16-dioxydi(meth)acrylate (isomer mixture; CAS 72869-86
c. IPDI-UDMA: 1,5,5-trimethyl-1-[(2-methacryloyloxyethyl)carbamoylmethyl]-3-(2-methacryloyloxyethyl)carbamoylcyclohexane (CAS 42405-01-6)
d. TEDMA: triethyleneglycol dimethacrylate (CAS 109-16-0)
e. DDDMA: 1,10-decanediol dimethacrylate (CAS 6701-13-9)
f. IBOMA: isobornyl methacrylate (CAS 7534-94-3)
g. PheMA: 2-phenoxyethyl methacrylate (CAS 10595-06-9)
h. DG1: dental glass G018-308 (0.7 μm; 3-MPS surface modification)
i. DG2: dental glass IS 50 1101 (0.4 μm; 3-MPS surface modification)
j. DG3: dental glass IS 50 1101 (1.0 μm; 3-MPS surface modification)
k. DG4: dental glass G018-308 (2.0 μm; 3-MPS surface modification)
l. PK1: pyrogenic silica (primary particles 12 nm; BET 145 m2/g; tamped density 188 g/L; HMDS surface modification; produced according to the example from DE 196 16 781 A1)
m. PK2: pyrogenic silica (primary particles 12 nm; BET 160 m2/g; tamped density 140 g/L; HMDS surface modification)
n. PK3: pyrogenic silica (primary particles 12 nm; BET 150 m2/g; tamped density 230 g/L; 3-MPS surface modification)
o. PK4: Aerosil A200 (primary particles 12 nm; BET 200 m2/g; tamped density 50 g/L)
p. PK5: Aerosil R816 (primary particles 12 nm; BET 190 m2/g; tamped density 60 g/L; HMDS surface modification)
q. HMDS: hexamethyldisilazane (CAS 999-97-3)
r. 3-MPS: 3-methacryloxypropyl(trimethoxy)silane (CAS 2530-85-0)
s. TPO: diphenyl(2,4,6-trimethylbenzoyl) phosphine oxide (CAS 75980-60-8)
t. BHT: 2,6-di-tert-butyl-4-methylphenol (CAS 128-37-0)
   Methods of measurement:

Flexural strength (FS): Flexural strengths were determined according to ISO 4049:2009. Test specimens having dimensions of 25 mm×2 mm×2 mm were produced from the printing resins by 3D printing (SolFlex350, W2P Engineering GmbH; wavelength 385 nm, power 8.3 mW/cm2, pixel size 50 μm, layer thickness 50 μm) and post-irradiated with 2×2000 flashes using an Otoflash instrument (VOCO GmbH). Determination of flexural strength is carried out at an advancement rate of 1 mm/min using a Zwick universal tester (Zwick GmbH & Co. KG, Ulm).

Elastic modulus: The elastic modulus was determined analogously to the calculation in ADA Spec. No. 27:1993 (7.8.4.2) as the gradient of the stress-strain curve of the 3-point flexural strength measurement in the linear-elastic region.

$$E = \frac{3 L}{4 b {h}^{3}} \frac{Δ F}{Δ d}$$

(L: span width; b:sample width; h:sample thickness; Ad:deformation in the linear-elastic region; ΔF: force change at deformation Ad)

Viscosity: Viscosity was measured using a Physica MCR 301 rheometer (Anton Paar, Graz) with a 50 mm measuring plate (plate/plate), 0.5 mm gap size and 1 g of substance. Before measurement the plate was temperature-controlled to a temperature of 23° C. The duration of measurement was 30 seconds at a shear rate of 10/s.

tan δ: The loss factor tan 8 (G″/G′) was determined at 23° C. using a Physica MCR 301 rheometer (Anton Paar, Graz) with a 50 mm measuring plate (cone/plate; angle 1°), 0.10 mm gap size and 0.2 g of substance. The deformation (deflection) was increased logarithmically from 0.0001% to 10% at 10 rad/s. 6 measurement points were recorded per decade. The measurement points were evaluated at 0.01% and 1% deformation.

Centrifuge stability:To assess centrifuge stability 3 g of the respective compositions were weighed into a centrifuge tube having a diameter of 15 mm and a length of 100 mm in a laboratory standard Rettich “EBA 20” centrifuge. The compositions were subsequently centrifuged three times for 5 minutes at 6000 rpm. The temperature during the centrifuge runs was 23° C.

Particle size: The particle size distribution of the fillers B1 was determined by static light scattering using a Beckman Coulter LS 13 320 instrument. The reported particle sizes are the d50 values with volume-weighted evaluation.

Production of 3D printing compositions: Production of the corresponding compositions comprised initially weighing in the monomers, initiators and inhibitors in the ratios reported below and in each case, stirring the resulting mixtures for two hours at room temperature until the solids had completely dissolved and homogeneous solutions were formed. The fillers (dental glass, pyrogenic silica, colour pigments) were then added in the reported ratios. The mixtures were then homogenized four times at 1500 rpm for 30 seconds in each case using a SpeedMixer DAC 800 apparatus (Hauschild GmbH & Co. KG, Hamm). The mixtures were subsequently deaerated at 1000 rpm and -0.9 bar for a further 30 seconds using the SpeedMixer DAC 800 apparatus.

TABLE 1

| Example | | 1 | 2 | 3 | 4 |
|---|---|---|---|---|---|
| A1 | Bis-EMA-4 | 45.0 | 45.0 | 45.0 | 45.0 |
| A2 | TMDI-UDMA | 20.0 | | 20.0 | |
| | IPDI-UDMA | | 20.0 | | 20.0 |
| A3 | IBOMA | 7.3 | 7.3 | | |
| | PheMA | | | 7.3 | 7.3 |
| B1 | DG1 | 20.0 | 20.0 | 20.0 | 20.0 |
| B2 | PK1 | 6.0 | 6.0 | 6.0 | 6.0 |
| C | TPO | 1.5 | 1.5 | 1.5 | 1.5 |
| D | BHT | 0.1 | 0.1 | 0.1 | 0.1 |
| E | TiO₂ | 0.1 | 0.1 | 0.1 | 0.1 |

| | | | | |
|---|---|---|---|---|
| Flexural strength | 107 | 109 | 103 | 105 |
| [MPa] | | | | |
| Elastic modulus | 3.3 | 3.3 | 3.1 | 3.1 |
| [GPa] | | | | |
| Viscosity (10/s) | 4500 | 4600 | 4650 | 4700 |
| [mPa*s] | | | | |
| tan δ (0.01%) | 0.82 | 0.82 | 0.84 | 0.85 |
| tan δ (1%) | 1.51 | 1.52 | 1.46 | 1.45 |
| Centrifuge stability | OK | OK | OK | OK |

TABLE 2

| Example | | 5 | 6 | 7 | 8 |
|---|---|---|---|---|---|
| A1 | Bis-EMA-4 | 42.5 | 40.0 | 42.5 | 40.0 |
| A2 | IPDI-UDMA | 20.0 | 20.0 | 20.0 | 20.0 |
| A3 | IBOMA | 6.8 | 6.3 | 6.8 | 6.3 |
| A4 | TEDMA | 3.0 | 6.0 | | |
| | DDDMA | | | 3.0 | 6.0 |
| B1 | DG1 | 20.0 | 20.0 | 20.0 | 20.0 |
| B2 | PK1 | 6.0 | 6.0 | 6.0 | 6.0 |
| C | TPO | 1.5 | 1.5 | 1.5 | 1.5 |
| D | BHT | 0.1 | 0.1 | 0.1 | 0.1 |
| E | TiO₂ | 0.1 | 0.1 | 0.1 | 0.1 |

| | | | | |
|---|---|---|---|---|
| Flexural strength | 110 | 113 | 108 | 109 |
| [MPa] | | | | |
| Elastic modulus | 3.7 | 3.7 | 3.4 | 3.4 |
| [GPa] | | | | |
| Viscosity (10/s) | 4820 | 4940 | 4800 | 4910 |
| [mPa*s] | | | | |
| tan δ (0.01%) | 0.74 | 0.70 | 0.79 | 0.76 |
| tan δ (1%) | 1.67 | 1.66 | 1.59 | 1.59 |
| Centrifuge stability | OK | OK | OK | OK |

TABLE 3

| Example | | 9 | 10 | 11 | 12 |
|---|---|---|---|---|---|
| A1 | Bis-EMA-4 | 49.5 | 45.0 | 40.0 | 35.0 |
| A2 | IPDI-UDMA | 27.0 | 25.0 | 18.0 | 12.0 |
| A3 | IBOMA | 7.1 | 7.3 | 7.3 | 4.5 |
| A4 | TEDMA | | | 3.4 | 4.0 |
| B1 | DG1 | 10.7 | 15.0 | 23.0 | 33.3 |
| B2 | PK1 | 4.0 | 6.0 | 6.6 | 9.5 |
| C | TPO | 1.5 | 1.5 | 1.5 | 1.5 |
| D | BHT | 0.1 | 0.1 | 0.1 | 0.1 |
| E | TiO₂ | 0.1 | 0.1 | 0.1 | 0.1 |

| | | | | |
|---|---|---|---|---|
| Flexural strength | 100 | 101 | 115 | 123 |
| [MPa] | | | | |
| Elastic modulus | 2.5 | 2.9 | 4.2 | 4.8 |
| [GPa] | | | | |
| Viscosity (10/s) | 2520 | 3130 | 5550 | 6000 |
| [mPa*s] | | | | |
| tan δ (0.01%) | 0.91 | 0.89 | 0.63 | 0.60 |
| tan δ (1%) | 1.90 | 1.83 | 1.23 | 1.05 |
| Centrifuge stability | OK | OK | OK | OK |

TABLE 4

| Example | | 13 | 14 | 15 | 16 |
|---|---|---|---|---|---|
| A1 | Bis-EMA-4 | 45.0 | 45.0 | 45.0 | 45.0 |
| A2 | IPDI-UDMA | 20.0 | 20.0 | 20.0 | 20.0 |
| A3 | IBOMA | 7.3 | 7.3 | 7.3 | 7.3 |
| B1 | DG1 | 20.0 | 20.0 | 20.0 | 20.0 |
| B2 | PK2 | 6.0 | | | |
| | PK3 | | 6.0 | | |
| | PK4 | | | 6.0 | |
| | PK5 | | | | 6.0 |
| C | TPO | 1.5 | 1.5 | 1.5 | 1.5 |
| D | BHT | 0.1 | 0.1 | 0.1 | 0.1 |
| E | TiO₂ | 0.1 | 0.1 | 0.1 | 0.1 |

| | | | | |
|---|---|---|---|---|
| Flexural strength | 110 | 108 | 98 | 99 |
| [MPa] | | | | |
| Elastic modulus | 3.3 | 3.3 | 3.1 | 3.1 |
| [GPa] | | | | |
| Viscosity (10/s) | 4620 | 4650 | 4640 | 4650 |
| [mPa*s] | | | | |
| tan δ (0.01%) | 0.83 | 0.84 | 1.05 | 1.08 |
| tan δ (1%) | 1.52 | 1.51 | 1.52 | 1.50 |
| Centrifuge stability | OK | OK | * | * |

* still OK after two runs; slight sedimentation detectable after third run

TABLE 5

| | | Example | | | | |
|---|---|---|---|---|---|---|
| | | 17 | 18 | 19 | 20 | 21 |
| A1 | Bis-EMA-4 | 45.0 | 45.0 | 45.0 | 45.0 | 45.0 |
| A2 | IPDI-UDMA | 20.0 | 22.0 | 19.0 | 19.0 | 20.0 |
| A3 | IBOMA | 7.3 | 7.3 | 7.3 | 7.3 | 7.3 |
| B1 | DG2 | 20.0 | 18.0 | | | |
| | DG3 | | | 21.0 | 21.0 | |
| | DG4 | | | | | 20.0 |
| B2 | PK1 | 6.0 | 6.0 | 6.0 | 6.0 | 6.0 |
| C | TPO | 1.5 | 1.5 | 1.5 | 1.5 | 1.5 |
| D | BHT | 0.1 | 0.1 | 0.1 | 0.1 | 0.1 |
| E | TiO₂ | 0.1 | 0.1 | 0.1 | 0.1 | 0.1 |

| | | | | | |
|---|---|---|---|---|---|
| Flexural strength | 118 | 111 | 102 | 106 | 1101 |
| [MPa] | | | | | |
| Elastic modulus | 3.9 | 3.5 | 2.9 | 3.1 | 3.1 |
| [GPa] | | | | | |
| Viscosity (10/s) | 5100 | 4920 | 2970 | 3110 | 3750 |
| [mPa*s] | | | | | |
| tan δ (0.01%) | 0.67 | 0.72 | 0.92 | 0.89 | 0.95 |
| tan δ (1%) | 1.22 | 1.30 | 1.88 | 1.69 | 1.03 |
| Centrifuge | OK | OK | OK | OK | * |
| stability | | | | | |

* still OK after two runs; slight sedimentation detectable after third run

TABLE 6

| | | 22 | 23 | 24 | 25 |
|---|---|---|---|---|---|
| Example | | (comparative) | (comparative) | (comparative) | (comparative) |
| A1 | Bis-EMA- | 45.0 | 49.8 | 55.0 | 45.0 |
| | 4 | | | | |
| A2 | IPDI- | 25.0 | 20.0 | 30.0 | 20.0 |
| | UDMA | | | | |
| A3 | IBOMA | 6.5 | 6.5 | 4.3 | 6.8 |
| B1 | DG1 | 20.0 | 10.0 | 7.0 | 26.5 |
| B2 | PK1 | 1.8 | 12.0 | 2.0 | |
| C | TPO | 1.5 | 1.5 | 1.5 | 1.5 |
| D | BHT | 0.1 | 0.1 | 0.1 | 0.1 |
| E | TiO₂ | 0.1 | 0.1 | 0.1 | 0.1 |

| | | | | |
|---|---|---|---|---|
| Flexural | 97 | 88 | 83 | 99 |
| strength [MPa] | | | | |
| Elastic modulus | 2.1 | 1.8 | 1.4 | 2.3 |
| [GPa] | | | | |
| Viscosity (10/s) | 2080 | 1920 | 1450 | 1820 |
| [mPa*s] | | | | |
| tan δ (0.01%) | 1.05 | 1.24 | 1.33 | 1.26 |
| tan δ (1%) | 1.89 | 2.20 | 2.95 | 2.30 |
| Centrifuge | no** | no** | no** | no*** |
| stability | | | | |

**material already sedimented after one run.
***material already sedimented after one run. In addition, sedimentation is already detectable after storage of two weeks at 23° C.

The invention is defined in the accompanying claims. Preferred aspects of the present invention are moreover derivable from the description which follows, including the examples.

Where particular embodiments are described as being preferred for one aspect of the invention (composition; cured product; therapeutic application) the corresponding intimations are in each case also applicable, mutatis mutandis, to the other aspects of the present invention. Preferred individual features of aspects of the invention (as defined in the claims and/or disclosed in the description) are combinable with one another and are preferably combined with one another unless the opposite is apparent to the person skilled in the art from the present text in the specific case.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A free-radically polymerizable composition for 3D printing of dental crowns, bridges, prosthetic teeth or full prostheses comprising:
A) a total amount of free-radically polymerizable (meth)acrylic compounds in an amount of 50% to 89% by weight,
B) a total amount of inorganic fillers in an amount of 10% to 49% by weight, and
C) a total amount of photoinitiators in an amount of 0.1% to 5% by weight,
in each case based on the total mass of the polymerizable composition, wherein the total amount (B) of inorganic fillers comprises
a first sub-amount (B1) of dental glass, and
a second sub-amount (B2) of pyrogenic silica,
and wherein the mass ratio of B1 to B2 is in the range from 10:1 to 1:1.

2. The free-radically polymerizable composition for 3D printing of dental crowns, bridges, prosthetic teeth or full prostheses according to claim 1, wherein the mass ratio of B1 to B2 is in the range from 5:1 to 1.5:1.

3. The free-radically polymerizable composition for 3D printing of dental crowns, bridges, prosthetic teeth or full prostheses according to claim 1, comprising:
A) in an amount of 56% to 85% by weight,
B) in an amount of 10% to 40% by weight, and
C) in an amount of 0.1% to 4% by weight,
in each case based on the total mass of the polymerizable composition.

4. The free-radically polymerizable composition for 3D printing of dental crowns, bridges, prosthetic teeth or full prostheses according to claim 1, wherein the dental glass B1
has an average particle size in the range from 0.4 to 5 μm, and/or
is selected from the group consisting of barium silicate glasses, barium aluminum silicate glasses, barium borosilicate glasses, barium boroaluminum silicate glasses, barium borofluoroaluminum silicate glasses, strontium silicate glasses, strontium aluminum silicate glasses, strontium borosilicate glasses, strontium boroaluminum silicate glasses, strontium borofluoroaluminum silicate glasses and zirconium silicate glasses, preferably selected from the group consisting of barium aluminum silicate glasses, barium borosilicate glasses and barium boroaluminum silicate glasses, and/or
is organically surface-modified.

5. The free-radically polymerizable composition for 3D printing of dental crowns, bridges, prosthetic teeth or full prostheses according to claim 1, wherein the pyrogenic silica B2 has a primary particle size in the range from 5 to 25 nm.

6. The free-radically polymerizable composition for 3D printing of dental crowns, bridges, prosthetic teeth or full prostheses according to claim 1, wherein the pyrogenic silica B2 is organically surface-modified.

7. The free-radically polymerizable composition for 3D printing of dental crowns, bridges, prosthetic teeth or full prostheses according to claim 1, wherein the pyrogenic silica B2 has a BET surface area in the range from 100 to 300 m2/g.

8. The free-radically polymerizable composition for 3D printing of dental crowns, bridges, prosthetic teeth or full prostheses according to claim 1, wherein the pyrogenic silica B2 has a tamped density in the range from 100 to 300 g/L.

9. The free-radically polymerizable composition for 3D printing of dental crowns, bridges, prosthetic teeth or full prostheses according to claim 1, wherein the pyrogenic silica B2 is structure-modified and/or compressed and post-milled.

10. The free-radically polymerizable composition for 3D printing of dental crowns, bridges, prosthetic teeth or full prostheses according to claim 1, wherein the total amount (A) of polymerizable (meth)acrylic compounds comprises:
a first sub-amount (A1) of difunctional, aromatic (meth)acrylates,
a second sub-amount (A2) of difunctional (meth)acrylates which comprise one or more urethane groups, and
a third sub-amount (A3) of monofunctional (meth)acrylates.

11. The free-radically polymerizable composition for 3D printing of dental crowns, bridges, prosthetic teeth or full prostheses according to claim 10, comprising:
A1) in an amount of 20% to 60% by weight,
A2) in an amount of 10% to 40% by weight, and
A3) in an amount of 1% to 10% by weight
in each case based on the total mass of the polymerizable composition.

12. The free-radically polymerizable composition for 3D printing of dental crowns, bridges, prosthetic teeth or full prostheses according to claim 10, wherein
A1 is selected from the group consisting of 2,2-bis[4-(meth)acryloyloxyethoxyphenyl]propane, 2,2-bis[4-(meth)acryloyloxydiethoxyphenyl]propane, 2,2-bis[4-(meth)acryloyloxytriethoxyphenyl]propane, 2,2-bis[4-(meth)acryloyloxytetraethoxyphenyl]propane, 2,2-bis [4-(meth)acryloyloxypentaethoxyphenyl]propane, 2,2-bis[4-(meth)acryloyloxydipropoxyphenyl]propane, 2,2-bis[4-(meth)acryloyloxyethoxyphenyl]-2-[4-(meth)acryloyloxydiethoxyphenyl]propane, 2-[4-(meth)acryloyloxydiethoxyphenyl]-2-[4-(meth)acryloyloxytriethoxyphenyl]propane, 2-[4-(meth)acryloyloxydipropoxyphenyl]-2-[4-(meth)acryloyloxytriethoxyphenyl]propane, 2,2-bis[4-(meth)acryloyloxyisopropoxyphenyl]propane, ethoxylated bisphenol A di(meth)acrylate and propoxylated bisphenol A di(meth)acrylate, and/or
A2 is selected from the group consisting of 3,14-dioxa-4, 13-dioxo-5, 12-diazahexadecane 1, 16-dioxydi(meth)acrylate, 7,7,9-trimethyl-3, 14-dioxa-4, 13-dioxo-5, 12-diazahexadecane 1, 16-dioxydi(meth)acrylate, 7,9,9-trimethyl-3, 14-dioxa-4, 13-dioxo-5, 12-diazahexadecane 1, 16-dioxydi(meth)acrylate and 1,5,5-trimethyl-1-[(2-(meth)acryloyloxyethyl) carbamoylmethyl]-3-(2-(meth)acryloyloxyethyl)carbamoylcyclohexane (CAS 42405-01-6 methacrylate; CAS 42404-50-2 acrylate), and/or
A3 is selected from the group consisting of 2-hydroxyethyl (meth)acrylate, 2-hydroxypropyl (meth)acrylate, 3-hydroxypropyl (meth)acrylate, tetrahydrofurfuryl (meth)acrylate, 4-(meth)acryloylmorpholine, cyclohexyl (meth)acrylate, 3,3,5-trimethylcyclohexyl (meth)acrylate, 4-(1,1-dimethylethyl)cyclohexyl (meth)acrylate, isobornyl (meth)acrylate, (octahydro-4,7-methano-1H-indenyl)methyl (meth)acrylate, benzyl (meth)acrylate, 2-phenoxyethyl (meth)acrylate, 2-(2-phenoxyethoxy)ethyl (meth)acrylate, 2-[2-(2-phenoxyethoxy)ethoxy]ethyl (meth)acrylate, ethoxylated 2-penoxyethyl (meth)acrylate, 2-ethoxyethyl (meth)acrylate, 2-(2-ethoxyethoxy)ethyl (meth)acrylate, 2-[2-(2-ethoxyethoxy)ethoxy]ethyl (meth)acrylate, 2-methoxyethyl (meth)acrylate, 2-(2-methoxyethoxy)ethyl (meth)acrylate, 2-[2-(2-methoxyethoxy)ethoxy]ethyl (meth)acrylate, hexyl (meth)acrylate, 2-ethylhexyl (meth)acrylate, decyl (meth)acrylate, isodecyl (meth)acrylate, dodecyl (meth)acrylate, 2-hydroxy-3-(prop-2-enoyloxy)propyl 2-methyl-2-propylhexanoate and (5-ethyl-1,3-dioxan-5-yl)methyl (meth)acrylate.

13. The free-radically polymerizable composition for 3D printing of dental crowns, bridges, prosthetic teeth or full prostheses according to claim 1, wherein the viscosity of the composition
at a shear rate of 0.01 s⁻¹ is in the range from 100 to 500 Pa*s, and/or
at a shear rate of 0.1 s⁻¹ is in the range from 10 to 50 Pa*s and/or
at a shear rate of 1 s⁻¹ is in the range from 2 to 20 Pa*s and/or
at a shear rate of 10 s⁻¹ is in the range from 1 to 10 Pa*s.

14. The free-radically polymerizable composition for 3D printing of dental crowns, bridges, prosthetic teeth or full prostheses according to claim 1, wherein the quotient tan δ of the loss modulus G″ and the storage modulus G′ under oscillating shear stress of the composition
for deflections in the range from 0.001% to 0.01% is less than 1, and
for deflections in the range from 1% to 10% is greater than 1.

15. A dental crown, bridge, prosthetic tooth or full prosthesis produced by 3D printing of a free-radically polymerizable composition according to claim 1

16. (canceled)

17. The free-radically polymerizable composition for 3D printing of dental crowns, bridges, prosthetic teeth or full prostheses according to claim 1, comprising:
B1) in an amount of 5% to 44.5% by weight, and
B2) in an amount of 0.9% to 24.5% by weight,
in each case based on the total mass of the polymerizable composition.

18. The free-radically polymerizable composition for 3D printing of dental crowns, bridges, prosthetic teeth or full prostheses according to claim 1, comprising:
A) in an amount of 67% to 84% by weight,
B) in an amount of 15% to 30% by weight, and
C) in an amount of 0.5% to 3% by weight,
in each case based on the total mass of the polymerizable composition.

19. The free-radically polymerizable composition for 3D printing of dental crowns, bridges, prosthetic teeth or full prostheses according to claim 4, wherein the dental glass B1 is organically surface-modified with 3-methacryloxypropyl(trimethoxy)silane.

20. The free-radically polymerizable composition for 3D printing of dental crowns, bridges, prosthetic teeth or full prostheses according to claim 13, wherein the viscosity of the composition
at a shear rate of 0.01 s⁻¹ is in the range from 200 to 270 Pa*s, and/or
at a shear rate of 0.1 s⁻¹ is in the range from 30 to 40 Pats, and/or
at a shear rate of 1 s⁻¹ is in the range from 6.5 to 10.5 Pa*s, and/or
at a shear rate of 10 s⁻¹ is in the range from 2.5 to 6.0 Pa*s.

21. The free-radically polymerizable composition for 3D printing of dental crowns, bridges, prosthetic teeth or full prostheses according to claim 14, wherein the quotient tan δ of the loss modulus G″ and the storage modulus G′ under oscillating shear stress of the composition
for deflections in the range from 0.001% to 0.01% is in the range from 0.4 to 0.99, and
for deflections in the range from 1% to 10% is in the range from 1.01 to 5.
